SEQUENCE LISTING (11 sequences)

SEQ ID NO: 1 - protein, 262 aa, Artifical sequence
  Synthesized
  MISC_FEATURE (93)..(93): Xaa = Hydrogen bond donating amino acid, such as Arg, Lys, or His
  MISC_FEATURE (158)..(158): Xaa = Negatively charged amino acid, such as Asp or Glu
MNPYARGPNPTAASLEASAGPFTVRSFTVSRPSGYGAGTVYYPTNAGGTVGAIAIVPGYTARQSSIKWWGPRLASHGFVVITIDTNSTLDQPXSRSSQQMAALRQVASLNGTSSSPIYGKVDTARMGVMGWSMGGGGSLISAANNPSLKAAAPQAPWXSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 2 - protein, 262 aa, Ideonella sakaiensis
MNPYARGPNPTAASLEASAGPFTVRSFTVSRPSGYGAGTVYYPTNAGGTVGAIAIVPGYTARQSSIKWWGPRLASHGFVVITIDTNSTLDQPSSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 3 - protein, 262 aa, Artifical sequence
  Synthesized
  MISC_FEATURE (93)..(93): X1 = Arg
  MISC_FEATURE (158)..(158): X2 = Asp
MNPYARGPNPTAASLEASAGPFTVRSFTVSRPSGYGAGTVYYPTNAGGTVGAIAIVPGYTARQSSIKWWGPRLASHGFVVITIDTNSTLDQPRSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 4 - protein, 262 aa, Artifical sequence
  Synthesized
  MISC_FEATURE (93)..(93): X1 = Lys
  MISC_FEATURE (158)..(158): X2 = Asp
MNPYARGPNPTAASLEASAGPFTVRSFTVSRPSGYGAGTVYYPTNAGGTVGAIAIVPGYTARQSSIKWWGPRLASHGFVVITIDTNSTLDQPKSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 5 - protein, 262 aa, Artifical sequence
  Synthesized
  MISC_FEATURE (93)..(93): X1 = His
  MISC_FEATURE (158)..(158): X2 = Asp
MNPYARGPNPTAASLEASAGPFTVRSFTVSRPSGYGAGTVYYPTNAGGTVGAIAIVPGYTARQSSIKWWGPRLASHGFVVITIDTNSTLDQPHSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 6 - protein, 262 aa, Artifical sequence
  Synthesized
  MISC_FEATURE (93)..(93): X1 = Leu
  MISC_FEATURE (158)..(158): X2 = Asp
MNPYARGPNPTAASLEASAGPFTVRSFTVSRPSGYGAGTVYYPTNAGGTVGAIAIVPGYTARQSSIKWWGPRLASHGFVVITIDTNSTLDQPLSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 7 - protein, 262 aa, Artifical sequence
  Synthesized
  MISC_FEATURE (93)..(93): X1 = Arg
  MISC_FEATURE (158)..(158): X2 = Glu
MNPYARGPNPTAASLEASAGPFTVRSFTVSRPSGYGAGTVYYPTNAGGTVGAIAIVPGYTARQSSIKWWGPRLASHGFVVITIDTNSTLDQPRSRSSQQMAALRQVASLNGTSSSPIYGKVDTARMGVMGWSMGGGGSLISAANNPSLKAAAPQAPWESSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 8 - protein, 261 aa, Artifical sequence
  Synthesized
  MISC_FEATURE (93)..(93): X1 = Lys
  MISC_FEATURE (158)..(158): X2 = Glu
MNPYARGPNPTAASLEASAGPFTVRSFTVSRPSGYGAGTVYYPTNAGGTVGAIAIVPGYTARQSSIKWWGPRLASHGFVVITIDTNSTLDQPKSRSSQQMAALRQVASLNGTSSSPIYGKVDTARMGVMGWSMGGGGSLISAANNPSLKAAAPQAPWESSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANC

SEQ ID NO: 9 - protein, 262 aa, Artifical sequence
  Synthesized
  MISC_FEATURE (93)..(93): X1 = His
  MISC_FEATURE (158)..(158): X2 = Glu
MNPYARGPNPTAASLEASAGPFTVRSFTVSRPSGYGAGTVYYPTNAGGTVGAIAIVPGYTARQSSIKWWGPRLASHGFVVITIDTNSTLDQPHSRSSQQMAALRQVASLNGTSSSPIYGKVDTARMGVMGWSMGGGGSLISAANNPSLKAAAPQAPWESSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 10 - protein, 262 aa, Artifical sequence
  Synthesized
  MISC_FEATURE (93)..(93): X1 = Leu
  MISC_FEATURE (158)..(158): X2 = Glu
MNPYARGPNPTAASLEASAGPFTVRSFTVSRPSGYGAGTVYYPTNAGGTVGAIAIVPGYTARQSSIKWWGPRLASHGFVVITIDTNSTLDQPLSRSSQQMAALRQVASLNGTSSSPIYGKVDTARMGVMGWSMGGGGSLISAANNPSLKAAAPQAPWESSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 11 - protein, 426 aa, Artificial Sequence
  Synthesized
  MISC_FEATURE (257)..(257): S to H
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAISDPNPYARGPNPTAASLEASAGPFTVRSFTVSRPSGYGAGTVYYPTNAGGTVGAIAIVPGYTARQSSIKWWGPRLASHGFVVITIDTNSTLDQPHSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

(19) United States
(12) Patent Application Publication
(10) Pub. No.: US 2024/0209331 A1
(43) Pub. Date: Jun. 27, 2024

(54) IDEONELLA SAKAIENSIS PET-HYDROLASE VARIANT WITH INCREASED THERMOSTABILITY

(71) Applicant: CycleZyme AB, Kävlinge (SE)

(72) Inventors: Peter Falck, Kävlinge (SE); Javier Antonino Linares-Patén, Lund (SE)

(21) Appl. No.: 18/043,179

(22) PCT Filed: Aug. 30, 2021
(86) PCT No.: PCT/EP2021/073865

§ 371 Date: Feb. 27, 2023

(30) Foreign Application Priority Data
Aug. 28, 2020 (EP) 20193259.7

(51) Int. Cl. C12N 9/18; C08J 11/10; C12P 7/18

(52) U.S. Cl. CPC C12N 9/18; C08J 11/105; C12P 7/18; C12Y 301/01

(57) ABSTRACT

The present disclosure relates to engineered polypeptides capable of degrading a polymer, such as polyethylene terephthalate (PET). The present disclosure also discloses polynucleotides encoding the polypeptides, vectors comprising the polynucleotides, as well as cells expressing the polynucleotides or vectors comprising the polynucleotides. Disclosed are also methods of degrading polymers, such as PET, and methods of manufacturing terephthalic acid and ethylene glycol.

### CROSS REFERENCE TO RELATED APPLICATIONS

This application is a U.S. National Stage of International Application No: PCT/EP2021/073865 filed Aug. 30, 2021, and which depends from and claims priority to European Patent Application No: EP 20193259.7 filed Aug. 28, 2020, the entire contents of which are incorporated herein by reference.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created Sep. 5, 2023, is named “2023-09-05 Sequence Listing Corrected CYCL0001PA_ST25” and is 28,719 bytes in size.

### TECHNICAL FIELD

The present invention relates to polypeptides for use in degradation of polymers.

### BACKGROUND

Plastics are key materials in a wide variety of strategic sectors such as packaging, transportation, medical devices, building & constructions, and others. World plastic production has increased from 299 Mt in 2013 to 335 Mt in 2016. Europe alone stands for 19% of the global production. There is an increased concern regarding accumulation of plastics in the environment, especially in marine ecosystems (Law, 2017). Plastics are highly stable and are expected to persist in the environment from hundreds to thousands of years.

Biodegradation is not yet a viable strategy for remediation or recycling. However, a recent study showed that the bacterium (Ideonella sakaiensis), which degrades and assimilates poly(ethylene terephthalate) (PET), was discovered in samples of a bottle recycling site (Yoshida et al., 2016). PET polymers are widely used in the manufacture of light weight containers for carbonated and non-carbonated drinks, juice, water, jellies, marmalades, and other similar foodstuffs. The enzyme involved in the hydrolysis of PET hydrolysis was identified as PET hydrolase (PETase), and its molecular structure has been elucidated (Han et al., 2017). Thus, the biodegradation of PET is a promising way for a green and sustainable process of recycling.

An effective application of PETase in PET recycling would be very beneficial, and requires higher temperatures than room temperature. This in turn requires a higher kinetic stability of the enzyme. It was recently reported that the β6-β7 connecting loop in PETase plays an important role in PETase stability and a variant with three mutations S121E/D186H/R280A showed substantially higher thermal stability and degradation activity in comparison with the wild type PETase (Son et al., 2019). Son et al., 2019 hypothesized that mutations of these positions in the PETase would lead to stabilization of the β6-β7 connecting loop by formation of hydrogen bonds, but failed to achieve this stabilization in any of the disclosed variants. The higher thermostability observed was instead due to interactions to residues elsewhere than the β6-β7 connecting loop.

PETases have the ability to substantially decrease the amount of time that it takes to degrade plastics, thereby making biodegradation a viable option for plastic recycling. Accordingly, there is a need in the art for PETases with higher thermostability and activity.

### SUMMARY

The inventors of the present invention has developed new mutant variants of PETase originally isolated from Ideonella sakaiensis (hereafter referred to as IsPETase), with higher thermostability and activity compared to other mutant and wild-type PETases. The high thermostability is proposed to be due to a single mutation which is capable of stabilizing the β6-β7 connecting loop by forming a stabilising interaction, such as a salt bridge, thus providing a solution to a problem that prior art has failed to solve. Therefore, the improved mutants as described herein carry high potential for industrial PET recycling as well as in new bio-based recycling methods of polyesters.

In one aspect of the present invention, the invention provides for a polypeptide comprising or consisting of the amino acid sequence SEQ ID NO: 1,

(SEQ ID NO: 1)
MNPYARGPNPTAASLEASAGPFTVRSFTVSRPSGYGAGTVYYPTNAGGT
VGAIAIVPGYTARQSSIKWWGPRLASHGFVVITIDTNSTLDQPX¹SRSS
QQMAALRQVASLNGTSSSPIYGKVDTARMGVMGWSMGGGGSLISAANNP
SLKAAAPQAPWDSSTNFSSVTVPTLIFACENDSIAPVNSSALPIYDSMS
RNAKQFLEINGGSHSCANSGNSNQALIGKKGVAWMKRFMDNDTRYSTFA
CENPNSTRVSDFRTANCS,

or a sequence having at least 70% identity to SEQ ID NO: 1,
wherein X¹ is a hydrogen bond-donating amino acid, and
wherein the polypeptide is capable of degrading polyester.

In another aspect of the present invention, the invention provides for a polypeptide comprising or consisting of the amino acid sequence SEQ ID NO: 1,

(SEQ ID NO: 1)
MNPYARGPNPTAASLEASAGPFTVRSFTVSRPSGYGAGTVYYPTNAGGT
VGAIAIVPGYTARQSSIKWWGPRLASHGFVVITIDTNSTLDQPX¹SRSS
QQMAALRQVASLNGTSSSPIYGKVDTARMGVMGWSMGGGGSLISAANNP
SLKAAAPQAPWX²SSTNFSSVTVPTLIFACENDSIAPVNSSALPIYDSM
SRNAKQFLEINGGSHSCANSGNSNQALIGKKGVAWMKRFMDNDTRYSTF
ACENPNSTRVSDFRTANCS,

or a sequence having at least 70% identity to SEQ ID NO: 1,
wherein X¹ is R, K, or H, and
wherein the side chain of the residue at position X² is negatively charged, and
wherein the polypeptide is capable of degrading polyester.

In one aspect of the present invention, the invention provides for a composition comprising said polypeptide.

In another aspect of the present invention, the invention provides for a polynucleotide encoding the polypeptide as described above.

In yet another aspect, the invention provides for a vector comprising said polynucleotide.

In one aspect, the invention provides for a cell comprising said polynucleotide or said vector.

In another aspect of the present invention, the invention provides for a method of degrading polyethylene terephthalate, the method comprising contacting the polypeptide, as described above, with a compound or composition comprising polyethylene terephthalate, thus degrading the polyethylene terephthalate.

In yet another aspect, the present invention provides for a method of manufacturing terephthalate acid, the method comprising the steps of:
(a) providing the polypeptide as described above,
(b) providing a material comprising polyethylene terephthalate,
(c) contacting the polypeptide of step (a) and the material comprising polyethylene terephthalate of (b), thus forming a mixture comprising terephthalic acid, and
(d) extracting terephthalic acid from said mixture, thus manufacturing terephthalic acid.

In another aspect, the present invention provides for a method of manufacturing ethylene glycol, the method comprising the steps of:
(a) providing the polypeptide as described above,
(b) providing a material comprising polyethylene terephthalate,
(c) contacting the polypeptide of step (a) and the material comprising polyethylene terephthalate of (b), thus forming a mixture comprising ethylene glycol, and
(d) extracting ethylene glycol from said mixture, thus manufacturing ethylene glycol.

### DESCRIPTION OF DRAWINGS

FIG. 1. Mutant IsPETase variants show higher thermostability compared to wild-type PETases. The figure shows the two molecular structures of the mutant variants of IsPETase with the highest thermostability; (A) S93K and (B) S93R. The indicated amino acid residues show an interaction between the residue at position 93 and the negatively charges amino acid aspartic acid in position 158, which resides in the β6-β7 connecting loop.

FIG. 2. Computational models of PETase and PETase-trx. The figure shows the molecular structures of wild-type PETase, with a fragment of PET (A), and the fusion protein PETase-trx (B). The fusion protein consists of a catalytic domain of wild-type PETase with a PET fragment, linked to the thioredoxin (trx) domain through a linker peptide.

FIG. 3. Purification analysis of PETase and PETase-trx. The figure shows a purified Immobilized Metal Affinity Chromatography (IMAC) purification chromatogram of PETase (A), an IMAC purification chromatogram of PETase-txr (B), where the large peaks to the left of each chromatogram represents proteins that did not bind to the column, and the peaks marked with arrows represent the elution of PETase and PETase-trx, respectively. PETase-trx clearly exhibits a larger peak, indicating a higher production yield compared to wild-type PETase. (C) shows a SDS-PAGE gel analysis 10 of PETase and PETase-trx where the molecular marker can be seen in lane M, recombinant PETase in lane (1) and PETase-trx in lane (2). The largest band can be observed in lane (2), supporting the conclusion of a higher production yield of PETase-trx compared to PETase.

FIG. 4. Relative activities of disclosed of PETase variants. The mutated variants SK and SR compared with the wild type (wt), where S is the serine residue changed by lysine (K) and arginine (R) respectively. All reactions were performed at 30° C., with the same amount of enzyme (0.003 mg) in 200 uL of reactions volume, pH 7 adjusted with 20 mM phosphate buffer. Activity of SK mutant: 100% (index); SR mutant: 92%; wild-type: 44%.

FIG. 5. Molecular dynamic simulations of Trx-S93H. The mutant S93H with Trx fusion domain has shown a slightly lower RMSD (FIG. 5A) compared with the wild type PETase. The gyration radii (FIG. 5B) also have a slightly lower average than the wild type PETase. All these results and the RMSF (FIG. 5C) analysis of the backbone have shown stabilizing effects on the region around the position 93, involved in the thermostability of the enzyme.

### DETAILED DESCRIPTION

The inventors of the present disclosure has identified new mutants of PETases. These PETases were originally identified in Ideonella sakaiensis. The new variants of the present disclosure exhibits activity on para-nitrophenyl butyrate after heating at 60° C. for 30 minutes, thereby exhibiting higher thermostability compared to previously identified variants and wild-type enzymes. PETases are esterases that catalyse the hydrolysis of PET plastics. The terms “PETase” and “PET hydrolase” are used interchangeably in the present document.

### Definitions

The terms “Ideonella sakaiensis” and “I. sakaiensis” are used herein interchangeably refer to a bacterium from the genus Ideonella and family Comamonadaceae (Betaprotobacteria) capable of breaking down and consuming the polymer poly ethylene terephthalate (PET) as a sole carbon and energy source. The bacterium was originally isolated from a sediment sample taken outside of a plastic bottle recycling facility in Sakai, Japan. The term “IsPETase” as used herein refers to PETase originally found in the bacterium Ideonella sakaiensis. The terms “IsPETase” and PETase are used interchangeably herein.

As used herein the singular forms “a”, “an” and “the” include plural aspects unless the context clearly dictates otherwise. Thus, for example, reference to “a mutation” includes a single mutation, as well as two or more mutations; reference to “a polypeptide” includes one polypeptide, as well as two or more polypeptides; and so forth.

As used herein “wild-type” refers to a full length protein comprised of a nucleic acid or amino acid sequence as would be found endogenously in a host cell or organism.

Reference to “mutation” refers to the presence of a different amino acid at a specified amino acid position within a protein sequence, respectively, than provided in a reference or wild-type sequence. The resulting mutation can be directly introduced into the reference or wild-type sequence or can be provided by a sequence other than the reference or wild-type sequence, as long as the end result provides for the indicated mutation.

The term “hydrogen bond” refers to a form of association between an electronegative atom (hereafter referred to as a “hydrogen bond acceptor”) and a hydrogen atom attached to a second, relatively electronegative atom (hereafter referred to as a “hydrogen bond donor”). A hydrogen bond donor further refers to an oxygen, nitrogen, or heteroaromatic carbon that bears a hydrogen group containing a ring of nitrogen or a heteroaryl group containing a ring nitrogen.

As used herein, the term “salt bridge” refers to the bond formed between oppositely charged residues, amino acids in a polypeptide or protein. In this disclosure, a salt bridge may be non-naturally occurring and introduced by way of point mutation.

The terms “X¹” and “X²” are used herein, to describe two specific positions of the sequences discloses herein. It is to be understood that X¹ and X² refer to positions S93 and D158, respectively, of SEQ ID NO: 2, and by way of analogy to the variants of SEQ ID NO: 2 disclosed herein. However, in certain prior art documents, these positions are instead referred to as S121 and D186, such as in Son et al., 2019. The latter system of counting takes into account the presence of a signal peptide at positions 1 to 28.

The term “hydrolysis” refers to any chemical reaction in which a molecule of water ruptures one or more chemical bonds, catalysed either by an enzyme or by a catalysator. The enzymatic hydrolysis is catalysed by a protease, which catalyses hydrolysis of internal peptide bonds in proteins/polypeptides, so enzymatic hydrolysis requires the presence of a protease. Hydrolysis of a protein may also be referred to as “proteolysis”, so any reference herein to “hydrolysis” of a proteinaceous material should be understood to include proteolysis. The proteolysis is enzymatic proteolysis.

With the term “terephthalic acid” is meant any of its salts or conjugate bases. That is, the terephthalic acid may be present in its uncharged form, in its singly charged form, i.e. as the monocarboxylate, in its doubly charged form i.e. the dicarboxylate, or in any of its salts such as a sodium salt, a disodium salt, and other salts with organic or inorganic counter ions.

### Polypeptides of the Disclosure

The present disclosure provides for mutants of PETase. The mutated amino acid residues are defined in SEQ ID NOs: 2-11.

The mutants of PETase disclosed herein may for example be useful for degradation of polyethylene terephthalate, such as for hydrolysis of polyethylene terephthalate. Degradation and or hydrolysis of polyethylene terephthalate preferably occurs at temperatures higher than room temperature, such as at a temperature of 30° C. or higher, such as at a temperature of 35° C. or higher, such as at a temperature of 40° C. or higher, such as at a temperature of 45° C. or higher, such as at a temperature of 50 ºC or higher, such as at a temperature of 55° C. or higher, such as at a temperature of 60° C. or higher, such as at a temperature in the range of 30° C. to 80° C., such as at a temperature in the range of 35° C. to 80 ºC, such as at a temperature in the range of 38° C. to 80 ºC, such as at a temperature in the range of 40° C. to 80° C., such as at a temperature in the range of 40° C. to 75 ºC, such as at a temperature in the range of 40° C. to 80° C.

It is thus advantageous that the mutants of PETase disclosed herein have better activity and stability at temperatures higher than room temperature compared to the wild type PETase.

In one embodiment of the present disclosure, the disclosure provides for a polypeptide having higher thermostability compared to the wild-type polypeptide.

In one embodiment of the present disclosure, the disclosure provides for a polypeptide having higher enzymatic activity compared to the wild-type polypeptide.

In one embodiment of the present disclosure, the disclosure provides for a polypeptide having higher enzymatic activity at temperatures higher than room temperature compared to the wild-type polypeptide.

In one embodiment of the present disclosure, the disclosure provides for a polypeptide having a stable enzymatic activity at temperatures in the range of 25° C. to 85° C., such as at temperatures in the range of 30° C. to 85 ºC, such as at temperatures in the range of 35° C. to 85° C., such as at temperatures in the range of 40° C. to 85° C., such as at temperatures in the range of 45° C. to 85° C., such as at temperatures in the range of 50 ºC to 85° C., such as at temperatures in the range of 50° C. to 85° C., such as at temperatures in the range of 55° C. to 85 ºC, such as at temperatures in the range of 60 ºC to 85° C., such as at temperatures in the range of 60° C. to 80° C.

A negatively charged amino acid residue at X² is preferred to enable interaction with the amino acid residue at X¹. X² may be a wild-type residue, or another negatively charged amino acid residue. Thus, X¹ is a residue capable of interacting with the negatively charged amino acid residue at X². It is therefore beneficial with amino acids that are hydrogen bond donors. The energy of a hydrogen bond can vary between 1 and 40 kcal/mol, depending on the nature of the donor and acceptor atoms constituting the bond, and their geometry and environment. The interaction between amino acid residues at X¹ and X² results in improved stability and function of the enzyme.

In one embodiment of the present disclosure, the disclosure provides for a polypeptide comprising or consisting of the amino acid sequence of SEQ ID NO: 1,

(SEQ ID NO: 1)
MNPYARGPNPTAASLEASAGPFTVRSFTVSRPSGYGAGTVYYPTNAGGT
VGAIAIVPGYTARQSSIKWWGPRLASHGFVVITIDTNSTLDQPX¹SRSS
QQMAALRQVASLNGTSSSPIYGKVDTARMGVMGWSMGGGGSLISAANNP
SLKAAAPQAPWDSSTNFSSVTVPTLIFACENDSIAPVNSSALPIYDSMS
RNAKQFLEINGGSHSCANSGNSNQALIGKKGVAWMKRFMDNDTRYSTFA
CENPNSTRVSDFRTANCS,

or a sequence having at least 70% identity to SEQ ID NO: 1,
wherein X¹ is a hydrogen bond-donating amino acid, and
wherein the polypeptide is capable of degrading polyester.

There are different hydrogen bond-donating amino acids, such as serine, threonine, histidine, lysine, arginine and tyrosine. Any of these may be suitable for the present disclosure. However, three of these are especially suitable for the present disclosure, by achieving improved thermostability, as can be seen in the Examples below. These amino acid residues have the ability to interact with the negatively charged amino acid residue at position X².

In another embodiment of the present disclosure, the invention provides for a polypeptide comprising or consisting of the amino acid sequence of SEQ ID NO: 1,

(SEQ ID NO: 1)
MNPYARGPNPTAASLEASAGPFTVRSFTVSRPSGYGAGTVYYPTNAGGT
VGAIAIVPGYTARQSSIKWWGPRLASHGFVVITIDTNSTLDQPX¹SRSS
QQMAALRQVASLNGTSSSPIYGKVDTARMGVMGWSMGGGGSLISAANNP
SLKAAAPQAPWX²SSTNFSSVTVPTLIFACENDSIAPVNSSALPIYDSM
SRNAKQFLEINGGSHSCANSGNSNQALIGKKGVAWMKRFMDNDTRYSTF
ACENPNSTRVSDFRTANCS,

or a sequence having at least 70% identity to SEQ ID NO: 1,
wherein X¹ is R, K, or H, and
wherein the side chain of the residue at position X² is negatively charged, and
wherein the polypeptide is capable of degrading polyester.

In one embodiment of the present disclosure, the sequence of said polypeptide has at least 75% identity to SEQ ID NO: 1, such as at least 80% identity to SEQ ID NO: 1, such as at least 85% identity to SEQ ID NO: 1, such as at least 90% identity to SEQ ID NO: 1, such as at least 95% identity to SEQ ID NO: 1, such as at least 96% identity to SEQ ID NO: 1, such as at least 97% identity to SEQ ID NO: 1, such as at least 98% identity to SEQ ID NO: 1, such as at least 99% identity to SEQ ID NO: 1.

In another embodiment of the present disclosure, said polypeptide comprises or consists of an amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11. In a preferred embodiment, the polypeptide comprises or consists of an amino acid sequence selected from the group consisting of: SEQ ID NO: 3, SEQ ID NO: 4, SEQ ID NO: 5 and SEQ ID NO: 11.

Arginine is a suitable amino acid residue for the present disclosure due to its ability to form a hydrogen bond to amino acid residue at position 158. The results as presented herein in the Examples show that arginine at position X¹ increases the activity and thermostability of the enzyme.

In one embodiment of the present disclosure, the amino acid residue of the X¹ position of said polypeptide is arginine.

Another amino acid with beneficial properties is lysine. It has the ability to form a hydrogen bond to amino acid residue at position 158. The results as presented herein in the Examples show that lysine at position X¹ increases the activity and thermostability of the enzyme.

In another embodiment of the present disclosure, the amino acid residue of the X¹ position of said polypeptide is lysine.

Histidine is also a suitable amino acid for the present disclosure, with beneficial results on the function of the enzyme.

In yet another embodiment of the present disclosure, the amino acid residue of the X¹ position of said polypeptide is histidine.

A negatively charged amino acid residue at X² is necessary to enable interaction with the amino acid residue at X¹. X² may be a wild-type residue, or another negatively charged amino acid residue. It is evaluated that the interaction between amino acid residues at X¹ and X² results in improved stability and function of the enzyme.

In one embodiment of the present disclosure, the amino acid residue at the X² position of said polypeptide is negatively charged.

It is advantageous with a residue at position X² to be a proteinogenic amino acid, since this would allow the residue to more feasibly be incorporated into the polypeptide when the polypeptide is produced in an in vivo system. Glutamic acid and aspartic acid are two such proteinogenic amino acids.

Therefore, in a further embodiment of the present disclosure, the amino acid residue at the X² position of said polypeptide is glutamic acid or aspartic acid.

A wild-type residue at the X² position could be advantageous, since this would eliminate the process of introducing an additional mutation.

In a further embodiment of the present disclosure, the amino acid residue at the X² position of said polypeptide is aspartic acid.

It has been suggested that the stability of the protein structure of IsPETases, and by virtue thereof the thermostability, is low due to a disruption of a centrally located β-sheet. This β-sheet consists of nine β-strands (β1-β9), wherein the β6 and β7 strands are connected by a connecting loop. In a study performed by Son et al., 2019, the authors attempts to stabilise the β6-β7 connecting loop by incorporating mutations to residues Ser121 and Asp186 (corresponding to S93 and Asp158 of the present disclosure, located in the β6-β7 connecting loop). However, the authors found that the distance between the residues, 3.9 Å, was too large to support the presence of a stabilising interaction, and accordingly, the combination of residues employed were insufficient to achieve stabilization of β6-β7 connecting loop. It is expected that the residues employed by the present disclosure, i.e. a negative residue at position X² and a hydrogen bond donating residue at position X¹ is sufficient to achieve stabilization of the β6-β7 connecting loop.

In one embodiment of the present disclosure, the distance between at least one atom in X¹ and at least one atom in X² is less than or equal to 5.0 Å, such as less than 4.5 Å, such as less than 4.0 Å, such as less than 3.9 Å, such as less than 3.8 Å, such as less than 3.7 Å, such as less than 3.6 Å, such as less than 3.5 Å. In another embodiment, the residues in positions X¹ and X² of said polypeptide are capable of interacting with each other. In yet another embodiment of the present disclosure, the residues at positons X¹ and X² interact with each other. In a further embodiment, said interaction is a charge-charge interaction. In an even further embodiment, said charge-charge interaction is a salt-bridge.

### Fusion to Thioredoxin

It may be useful to conjugate the polypeptide disclosed herein to thioredoxin. A fusion polypeptide of thioredoxin and the polypeptide as disclosed herein could be highly beneficial, in that it may results in higher solubility and stability in solution. The conjugation of the fusion polypeptide can be to the N-terminal or C-terminal end, or to a side-chain.

In one embodiment of the present disclosure, said polypeptide is conjugated to thioredoxin.

In another embodiment, thioredoxin is conjugated to the N-terminal of the polypeptide disclosed herein. For example, in one embodiment the polypeptide disclosed herein is SEQ ID NO:11.

In one embodiment of the present disclosure, thioredoxin is conjugated to the N-terminal of the polypeptide disclosed herein via a linker. For example, the linker may be an amino acid sequence, such as a small peptide.

In yet another embodiment, thioredoxin is conjugated to the C-terminal of the polypeptide disclosed herein.

In one embodiment, thioredoxin is conjugated to a side-chain of the polypeptide of the disclosure.

Polypeptides are often sold in buffer solutions, which increases stability and thereby shelf-life.

Therefore, in one embodiment of the present disclosure, the invention provides for a composition comprising the polypeptide as described herein.

In another embodiment, said composition is an aqueous buffer.

### Polynucleotides, Vectors, and Cells

The polypeptide enclosed herein can be produced in an organism or by translation and other useful methods. Hence, there is a need for a polynucleotide.

Accordingly, in one embodiment of the present disclosure, the invention provides for a polynucleotide encoding the polypeptide as described above.

One way to aid production in organisms, is to use vectors. They are extra useful in bacteria.

In another embodiment, the disclosure provides for a vector comprising said polynucleotide.

It may also be useful to have a purely biochemical production of the PETase as disclosed herein. Thus, there is need of a cell capable of producing the polypeptide of the present disclosure.

Therefore, in a further embodiment, the invention provides for a cell comprising said polynucleotide or said vector.

### Methods of Degrading Polyethylene Terephthalate

There is an increased concern regarding accumulation of plastics in the environment, especially in marine ecosystems (Law, 2017). Plastics are highly stable and are expected to persist in the environment from hundreds to thousands of years.

Due to the increasing accumulation of plastics in the environment, it is necessary to develop new and more rapid methods of degrade and recycle plastics, and in particular methods of degrading polyethylene terephthalate.

In another embodiment of the present disclosure, the invention provides for a method of degrading polyethylene terephthalate, the method comprising contacting the polypeptide, as described above, with a compound or composition comprising polyethylene terephthalate, thus degrading the polyethylene terephthalate.

If the degradation of a polymer is performed in a controlled manner, such as through hydrolysis, discrete products may be produced. That is, the monomeric units constituting the polymer may be recovered. Therefore, hydrolysis is advantageous. Hydrolysis effects cleavage of specific moieties within the polymer, such as esters or amides. Effective hydrolysis of polyethylene terephthalate provides a solution to the problem of accumulation of PET plastic in the nature.

In a preferred embodiment of the present disclosure, degradation means hydrolysis.

In one embodiment of the present disclosure, the degradation of the polyethylene terephthalate is hydrolysis of the polyethylene terephthalate.

In one embodiment of the present disclosure, the composition comprising polyethylene terephthalate is a composite material. A person skilled in the art will understand that the term “composite material” (also referred to as “composition material” or “composite”) refers to a material made from two or more constituent materials that when combined, produce a material different from the individual components. The individual components generally remain separate and distinct within the finished structure, differentiating composite materials from mixtures and solid solutions. The composite material can include, but is not limited to, textile, food packaging, plastics, and blends of PET/cellulose, PET/polypropylene and PET/polyethylene as well as blends comprising polyester and one of the following: cotton, cellulose, polypropylene, nylon, acrylics, wool and elastane. In one embodiment of the present disclosure, said composite material comprises at least one other polymer than polyethylene terephthalate.

In addition to degrading plastics, and thereby avoiding accumulation in the environment, repurposing of plastics is advantageous. This would allow for decreased production of plastics, and thereby less use of fossil fuels, and could also lower pollution. Therefore, a method of realising repurposing of plastics such as PET, by recovering the monomeric units of the polymer is sought-after.

In one embodiment of the present disclosure, the present invention provides for a method of manufacturing terephthalate acid, the method comprising the steps of:
(a) providing the polypeptide as described above,
(b) providing a material comprising polyethylene terephthalate,
(c) contacting the polypeptide of step (a) and the material comprising polyethylene terephthalate of step (b), thus forming a mixture comprising terephthalic acid, and
(d) extracting terephthalic acid from said mixture,
   thus manufacturing terephthalic acid.

In another embodiment, the present disclosure provides for a method of manufacturing ethylene glycol, the method comprising the steps of:
(a) providing the polypeptide as described above,
(b) providing a material comprising polyethylene terephthalate,
(c) contacting the polypeptide of step (a) and the material comprising polyethylene terephthalate of (b), thus forming a mixture comprising ethylene glycol, and
(d) extracting ethylene glycol from said mixture,
   thus manufacturing ethylene glycol.

By performing some or all of the steps in the process on degrading polyethylene terephthalate and thus manufacturing terephthalic acid or ethylene glycol at a temperature higher than room temperature, the reaction rate can be increased. It is therefore useful to have thermo-stable variants of the enzyme. The variants of the present disclosure are thermo-stable, even up to temperatures higher than 40° C.

In one embodiment of the present disclosure, said method further comprises a step wherein the mixture from step (c) is heated. In another embodiment, said mixture is heated to at least 45° C., such as at least 50° C., such as at least 55° C., such as at least 58° C., such as least 60° C.

The enzymes disclosed herein may also be useful in degrading other polyesters than PET.

In another embodiment, the polypeptide of said method is capable of degrading polyester. In yet another embodiment, the polypeptide of said method is capable of degrading polyethylene terephthalate.

In a further embodiment, the polypeptide of said method is capable of degrading a polymeric substrate at a temperature of at least 45° C., such as at least 50° C., such as at least 55° C., such as at least 58° C., such as at least 60° C. In a preferred embodiment, said polypeptide is capable of degrading polyethylene terephthalate at a temperature of at least 45° C., such as at least 50° C., such as at least 55° C., such as at least 58° C., such as at least 60° C., such as at 60° C.

Enzymes unfold/denature at certain temperatures. It is therefore advantageous that they are maintained in folded (and active) state even at a high temperature.

In one embodiment, the Tₘ of said polypeptide is at least 40° C., such as at least 45° C., such as at least 50° C., such as at least 52° C., such as at least 54° C., such as at least 55° C., such as at least 56° C., such as at least 58° C., such as at least 60° C.

Stability in solution is important, as long shelf life is advantageous. Stability in solution may also result in higher activity due to more available enzyme, as well as more efficient production since the enzyme produced will stay in solution for longer, i.e. not precipitate or aggregate.

In another embodiment, said polypeptide is stable in a solution for at least 5 months at 4ºC.

In yet another embodiment, said polypeptide is stable in a solution for at least 1 day, such as 2 days, such as 3 days, such as 4 days, such as 5 days, such as 6 days, such as 1 week, such as 2 weeks, such as 3 weeks, such as 4 weeks, such as 1 month, such as 2 months, such as 3 months, such as 4 months at 4° C.

As outlined in the background, it has been suggested that the stability of the protein structure of IsPETases, and by virtue thereof the thermostability, is low due to a disruption of a centrally located β-sheet. It is expected that the residues employed by the present disclosure may be capable of forming a disulphide bond, which could be able to achieve stabilization of the β6-β7 connecting loop.

In one embodiment of the present disclosure, the invention provides for a polypeptide comprising or consisting of the amino acid sequence of SEQ ID NO: 1,

(SEQ ID NO: 1)
MNPYARGPNPTAASLEASAGPFTVRSFTVSRPSGYGAGTVYYPTNAGGT
VGAIAIVPGYTARQSSIKWWGPRLASHGFVVITIDTNSTLDQPX¹SRSSQ
QMAALRQVASLNGTSSSPIYGKVDTARMGVMGWSMGGGGSLISAANNPS
LKAAAPQAPWX²SSTNFSSVTVPTLIFACENDSIAPVNSSALPIYDSMSR
NAKQFLEINGGSHSCANSGNSNQALIGKKGVAWMKRFMDNDTRYSTFAC
ENPNSTRVSDFRTANCS,

or a sequence having at least 70% identity to SEQ ID NO: 1,
wherein X¹ is cysteine (C), and
wherein X² is cysteine (C), and wherein the polypeptide is capable of degrading polyester.

In one embodiment, the cysteine moieties at X¹ and X² are capable of forming a disulphide bridge.

### EXAMPLES

### Example 1. Production and Mutagenesis of Recombinant IsPETase

### Methods

Production of recombinant IsPETase

The gene encoding recombinant PETase from I. sakaiensis (GenBank accession code GAP38373.1) without signal peptide (first 29 amino acids from the N-terminal) was chemically synthesized with codons optimized for Escherichia coli (E. coli), and cloned into the expression vector pET28b(+). The recombinant protein was produced in E. coli Rosetta-Gami™ 2 (DE3) (Novagen, U.S) a purified Immobilized Metal Affinity Chromatography (IMAC) with a HisTrap™ Fast Flow column installed in an ÄKTA start protein purification system (GE Healthcare Bio-Sciences AB Uppsala, Sweden) (Arza et al., 2019; Wang et al., 2020).

### Computational Studies

Crystallographic structure of IsPETase (Protein Data Bank, accession code 5XJH) (Joo et al., 2018) was used to generate mutants in the β6-β7 connecting loop. Both wild-type structure and mutants were subjected to molecular dynamic simulations at 37° C. and 60° C. for 500 ns.

### Site-Directed Mutagenesis

A point-mutation in position 93 of the β6-β7 connecting loop was introduced by site-directed mutagenesis using the plasmid encoding the wild-type gene as template (pET28b(+) IsPETase) and the QuikChange Site Directed Mutagenesis Kit, according to the instruction by the manufacturer (Stratagene, La Jolla, CA, USA). The mutants were produced and purified in the same way as the wild-type, as described above.

### Results

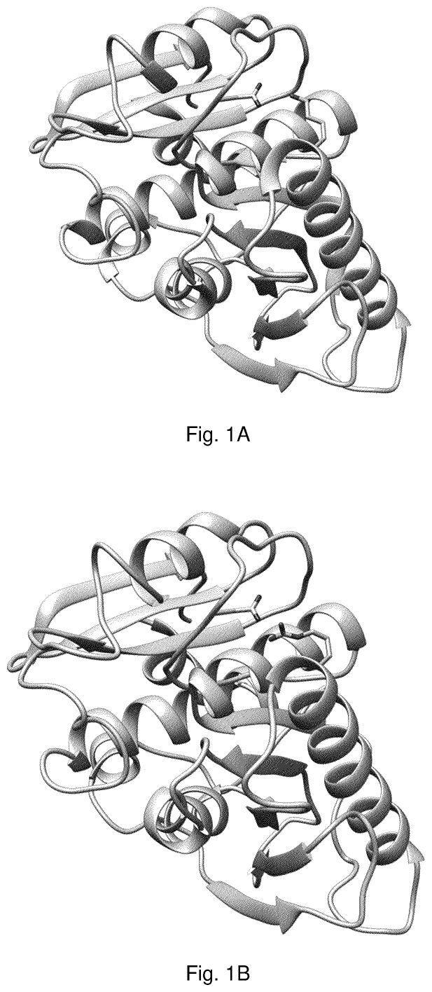

Both wild-type (SEQ ID NO: 2) and mutant enzymes, S93R (SEQ ID NO: 3), S93K (SEQ ID NO: 4) and S93L (SEQ ID NO: 6), were produced successfully. The mutant S93H (SEQ ID NO. 5), however, was not expressed in the E. coli bacteria. The short time molecular dynamic simulations have predicted that mutations S93R and S93K stabilize the β6-β7 connecting loop (FIG. 1).

### Conclusion

The inventors were able to successfully produce IsPETase mutants. Computational studies with short time molecular dynamic simulations have allowed the rational design of thermostable variants of IsPETase, introducing a single mutation at position 93.

### Example 2. Thermostability and Activity of Mutant and Wild-Type PETase

### Methods

### Enzyme Activity

The computer simulations indicated that variants S93R (SEQ ID NO. 3) and S93K (SEQ ID NO. 4) would have a stable β6-β7 connecting loop. These mutants, in addition to S93L (SEQ ID NO. 6), were therefore selected for experimental studies. The activity of both wild-type IsPETase and mutants, was assessed using para-nitrophenyl butyrate as substrate. Wild-type (SEQ ID NO. 2) and mutants were subjected to a heat treatment in a range of temperature from 37° C. to 60° C. for 30 minutes. Thereafter, residual activities were assessed in triplicates, using 5 mM of substrate and 50 mM phosphate buffer, pH 7. All reactions, including a control (same reaction components, except enzyme), were performed in the same conditions and monitored online at 410 nm of wavelength in a microplate spectrophotometer (Thermo Scientific™, Multiskan™ GO) during 30 min at 37° C.

### Results

Mutants selected after the computational studies were experimentally assessed with a chromogenic synthetic ester para-nitrophenyl butyrate. After heat treatment at 60° for 30 minutes, mutants S93R and S93K showed remarkable residual activity (FIG. 4), particularly S93K, while the wild-type and S93L mutant had slightly lower activity compared with heat treatment at 37° C. Both variants had considerably higher activity than the wild-type enzyme.

### Conclusion

The data suggests that S93K and S93R mutations have the ability to stabilize the β6-β7 connecting loop by the introduction of a salt bridge, thereby resulting in higher thermostability and activity of the mutant enzymes compared to wild-type enzymes. These new enzymes are promising candidates for applications in degradation and recycling of plastic material based on PET.

### Example 3. PETase as a Fusion Protein

### Methods

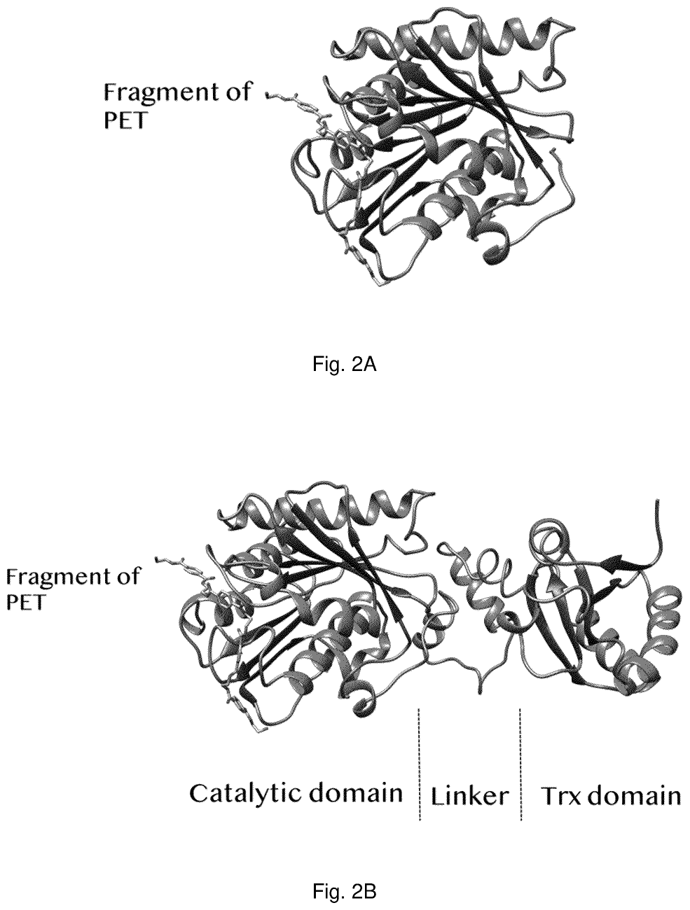

A catalytic domain of PETase was linked to a thioredoxin (txr) domain through a linker peptide (FIGS. 2A and B). A fragment of PET (DP4) was docked into the active site. This complex was subjected to 200 ns of molecular and dynamic simulations to investigate the effect of txr on wild-type PETase activity. Purification analyses were performed in order to compare the production yield of wild-type PETase and the PETase-trx fusion protein.

Furthermore, the degradation capacity of wild-type PETase compared to the PETase-trx fusion protein was studied. For the degradation profiles of PETase and PETase-trx, PET samples (20 mg) were soaked in a mixture of 50 mM phosphate buffer (1.0 mL, pH 7.2), DMSO (0.2 mL) and freshly prepared PETase enzyme (0.05 mL, 1.78 g/L) or Cut1b enzyme (0.05 mL, 1.99 g/L) adjusted with MilliQ water [5, 13, 17, 23]. The reaction mixtures were incubated at 37° C. and 200 rpm for 72 h. After re-suspending the content of the reactions, a sample of 0.5 mL of each was retrieved, diluted in a 1:1 ratio with DMSO, filtered (200 nm PTFE membrane) and transferred to HPLC tubes for further analysis. All reactions and negative controls (without enzyme) were performed in triplicates. All resulting peaks from the HPLC analysis were regarded as terephthalic acid equivalents (TPAeq) (due to the same molar extinction coefficient, e=17,000 M⁻¹ cm⁻¹).

### Results

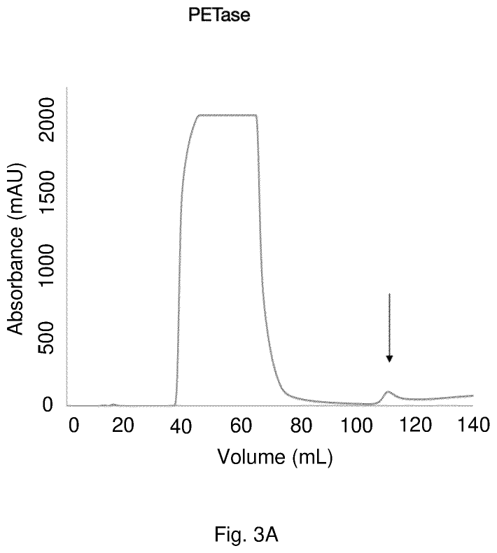

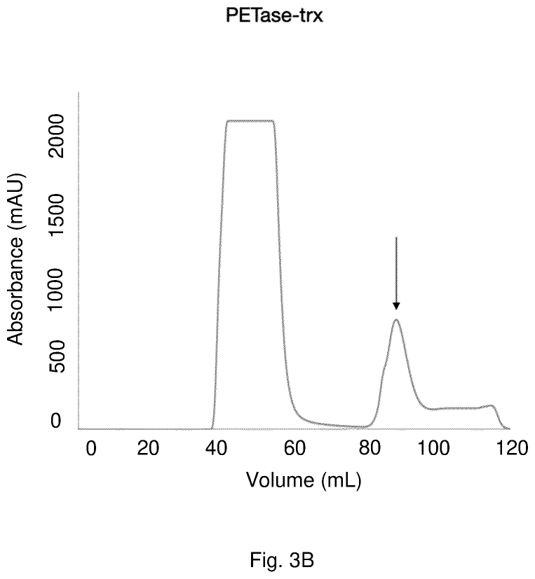

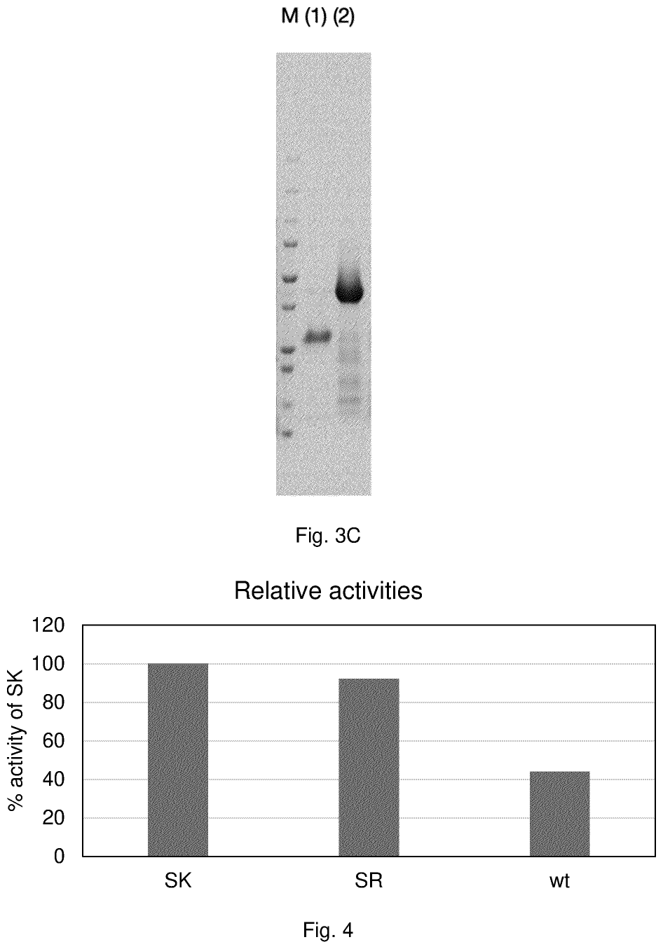

The molecular model of the fusion protein PETase-txr has shown that the fusion domain affects neither the active site nor the interaction with the substrate. The dynamic simulations suggested that the linker is flexible but not enough to generate conformers in which the trx domain affect negatively the catalytic site of the enzyme. Furthermore, PETase-trx was produced in significantly higher yield compared with the native PETase (FIG. 3). The PETase-trx fusion protein exhibited higher PET degradation compared to wild-type PETase, with 32.3% of all PET degraded compared to 23.04% for the wild-type enzyme (Table 1).

TABLE 1
Percentage of PET (20 mg) degraded with PETase and PETase-trx.

| | TPA equivalents (mg/L) | % of degradation |
|---|---|---|
| PET-ase (wild-type) | 2.304 | 23.04% |
| PETase-trx | 3.230 | 32.30% |

### Conclusion

The PETase-trx fusion protein was produced with a higher yield compared to PETase and showed an increased activity compared to PETase, indicating a potential use of this fusion protein in industrial degradation and recycling of PET plastics.

### Example 4. PETase Variant S93H with Trx Fusion Domain

### Materials and Methods

Molecular dynamic simulations. The mutant S93H with Trx fusion domain (Trx-S93H) was obtained as described in Examples 1 and 3, and it was subjected to molecular dynamics simulations with explicit molecules of water as a solvent. All calculations were performed in YASARA v 18.4.24 (Krieger and Vriend 2014), using AMBER14 force field (Case et al. 2014). A cubic simulation cell, 20 Å larger than the complex, with periodic boundary conditions, was filled with TIP3P water molecules and counter ions (Mark and Nilsson 2001). The distance for Van der Waals interactions was set in a medium range of 8 Å, and the long-range Coulomb forces were calculated using the particle-mesh Ewald algorithm (Darden et al. 1993). The temperature control was through the Berendsen Thermostat (Berendsen et al. 1984). The simulated conditions were: 0.9% NaCl, pH 9, 0.982 g mL-1 solvent density, and 60° C. during 25 ns, saving snapshots every 100 ps. Root-mean-square deviations (RMSD) of atomic coordinates, root-mean-square fluctuations (RMSF), and enzyme/ligand interactions were analysed.

### Results

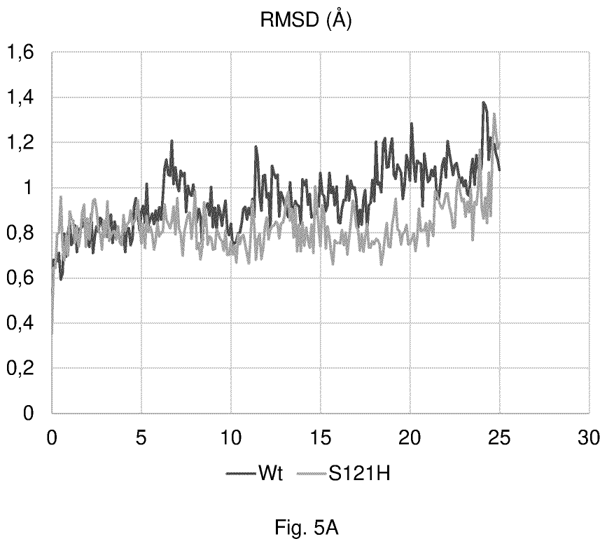

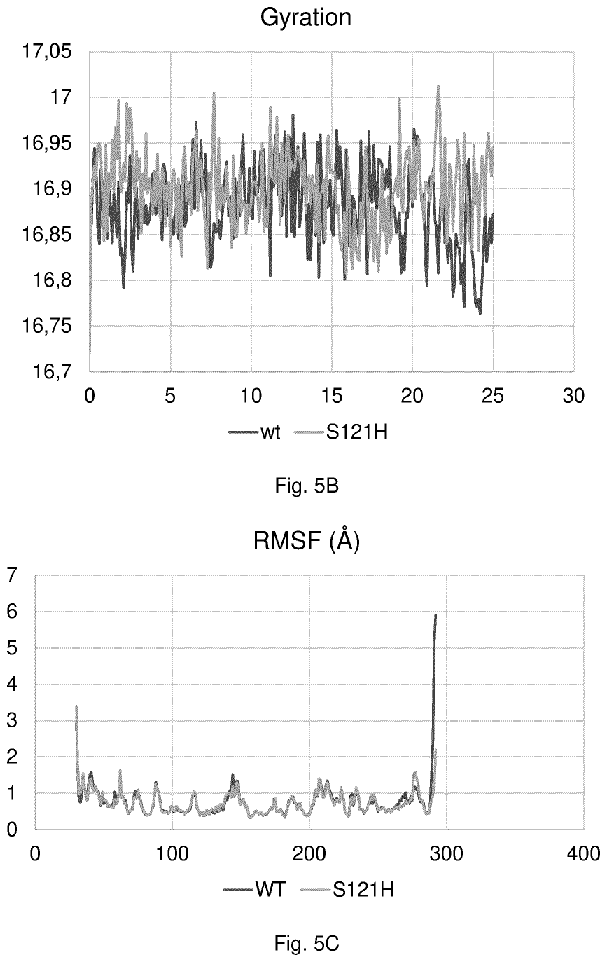

The mutant S93H with Trx fusion domain has shown a slightly lower RMSD (FIG. 5A) compared with the wild type PETase. The gyration radii (FIG. 5B) also have a slightly lower average than the wild type PETase. All these results and the RMSF (FIG. 5C) analysis of the backbone have shown for the first time stabilizing effects on the region around the position 93, involved in the thermostability of the enzyme. Experimentally, S93H activity tested with a small substrate pNP-butyrate at 40° C. and 80° C., has shown no substantial decrement, compared with the wild type PETase, however, lower activity than wild type PETase. In particular, the wild-type lost 71% of its activity at 80° C., while the variant Trx-S121H lost 59% of activity at the same temperature (compared to their activity at 40° C.). However, the wild-type had higher activity than the variant, 14 U/mg against 4 U/mg at 40° C., and 4 U/mg against 2 U/mg at 80° C.

The fusion Trx domain, additionally confers more stability in its production, decreasing the formation of inclusion bodies, and thereby made it possible to express the mutant S93H in E. coli.

### Conclusion

Molecular dynamic simulations and experimental test with pNP-butyrate suggest that the variant PETase Txr-S93H has a longer lifespan than wild type PETase at higher temperatures, although a lower activity. The stability conferred by this single mutation can be suitable for applications at low temperatures with long periods of time.

### Sequences

SEQ ID NO: 1: Markush sequence
MNPYARGPNPTAASLEASAGPFTVRSFTVSRPSGYGAGTVYYPTNAGGTVGAIAIVPGYTARQS
SIKWWGPRLASHGFVVITIDTNSTLDQPX¹SRSSQQMAALRQVASLNGTSSSPIYGKVDTARMG
VMGWSMGGGGSLISAANNPSLKAAAPQAPWX²SSTNESSVTVPTLIFACENDSIAPVNSSALPI
YDSMSRNAKQFLEINGGSHSCANSGNSNQALIGKKGVAWMKREMDNDTRYSTFACENPNSTRVS
DERTANCS
SEQ ID NO: 2: Wild-type
MNPYARGPNPTAASLEASAGPFTVRSFTVSRPSGYGAGTVYYPTNAGGTVGAIAIVPGYTARQS
SIKWWGPRLASHGFVVITIDTNSTLDQPSSRSSQQMAALRQVASLNGTSSSPIYGKVDTARMGV
MGWSMGGGGSLISAANNPSLKAAAPQAPWDSSTNESSVTVPTLIFACENDSIAPVNSSALPIYD
SMSRNAKQFLEINGGSHSCANSGNSNQALIGKKGVAWMKREMDNDTRYSTFACENPNSTRVSDF
RTANCS
SEQ ID NO: 3: S93R mutant
MNPYARGPNPTAASLEASAGPFTVRSFTVSRPSGYGAGTVYYPTNAGGTVGAIAIVPGYTARQS
SIKWWGPRLASHGFVVITIDTNSTLDQPRSRSSQQMAALRQVASLNGTSSSPIYGKVDTARMGV
MGWSMGGGGSLISAANNPSLKAAAPQAPWDSSTNESSVTVPTLIFACENDSIAPVNSSALPIYD
SMSRNAKQFLEINGGSHSCANSGNSNQALIGKKGVAWMKREMDNDTRYSTFACENPNSTRVSDE
RTANCS
SEQ ID NO: 4: S93K mutant
MNPYARGPNPTAASLEASAGPFTVRSFTVSRPSGYGAGTVYYPTNAGGTVGAIAIVPGYTARQS
SIKWWGPRLASHGFVVITIDTNSTLDQPKSRSSQQMAALRQVASLNGTSSSPIYGKVDTARMGV
MGWSMGGGGSLISAANNPSLKAAAPQAPWDSSTNFSSVTVPTLIFACENDSIAPVNSSALPIYD
SMSRNAKQFLEINGGSHSCANSGNSNQALIGKKGVAWMKREMDNDTRYSTFACENPNSTRVSDE
RTANCS
SEQ ID NO: 5: S93H mutant
MNPYARGPNPTAASLEASAGPFTVRSFTVSRPSGYGAGTVYYPTNAGGTVGAIAIVPGYTARQS
SIKWWGPRLASHGFVVITIDTNSTLDQPHSRSSQQMAALRQVASLNGTSSSPIYGKVDTARMGV
MGWSMGGGGSLISAANNPSLKAAAPQAPWDSSTNESSVTVPTLIFACENDSIAPVNSSALPIYD
SMSRNAKQFLEINGGSHSCANSGNSNQALIGKKGVAWMKREMDNDTRYSTFACENPNSTRVSDE
RTANCS
SEQ ID NO: 6: S93L mutant
MNPYARGPNPTAASLEASAGPFTVRSFTVSRPSGYGAGTVYYPTNAGGTVGAIAIVPGYTARQS
SIKWWGPRLASHGFVVITIDTNSTLDQPLSRSSQQMAALRQVASLNGTSSSPIYGKVDTARMGV
MGWSMGGGGSLISAANNPSLKAAAPQAPWDSSTNESSVTVPTLIFACENDSIAPVNSSALPIYD
SMSRNAKQFLEINGGSHSCANSGNSNQALIGKKGVAWMKREMDNDTRYSTFACENPNSTRVSDE
RTANCS
SEQ ID NO: 7: S93R/D158E mutant
MNPYARGPNPTAASLEASAGPFTVRSFTVSRPSGYGAGTVYYPTNAGGTVGAIAIVPGYTARQS
SIKWWGPRLASHGFVVITIDTNSTLDQPRSRSSQQMAALRQVASLNGTSSSPIYGKVDTARMGV
MGWSMGGGGSLISAANNPSLKAAAPQAPWESSTNESSVTVPTLIFACENDSIAPVNSSALPIYD
SMSRNAKQFLEINGGSHSCANSGNSNQALIGKKGVAWMKREMDNDTRYSTFACENPNSTRVSDE
RTANCS
SEQ ID NO: 8: S93K/D158E mutant
MNPYARGPNPTAASLEASAGPFTVRSFTVSRPSGYGAGTVYYPTNAGGTVGAIAIVPGYTARQS
SIKWWGPRLASHGFVVITIDTNSTLDQPKSRSSQQMAALRQVASLNGTSSSPIYGKVDTARMGV
MGWSMGGGGSLISAANNPSLKAAAPQAPWESSTNESSVTVPTLIFACENDSIAPVNSSALPIYD
SMSRNAKQFLEINGGSHSCANSGNSNQALIGKKGVAWMKREMDNDTRYSTFACENPNSTRVSDE
RTANCS
SEQ ID NO: 9: S93H/D158E mutant
MNPYARGPNPTAASLEASAGPFTVRSFTVSRPSGYGAGTVYYPTNAGGTVGAIAIVPGYTARQS
SIKWWGPRLASHGFVVITIDTNSTLDQPHSRSSQQMAALRQVASLNGTSSSPIYGKVDTARMGV
MGWSMGGGGSLISAANNPSLKAAAPQAPWESSTNESSVTVPTLIFACENDSIAPVNSSALPIYD
SMSRNAKQFLEINGGSHSCANSGNSNQALIGKKGVAWMKREMDNDTRYSTFACENPNSTRVSDE
RTANCS
SEQ ID NO: 10: S93L/D158E mutant
MNPYARGPNPTAASLEASAGPFTVRSFTVSRPSGYGAGTVYYPTNAGGTVGAIAIVPGYTARQS
SIKWWGPRLASHGFVVITIDTNSTLDQPLSRSSQQMAALRQVASLNGTSSSPIYGKVDTARMGV
MGWSMGGGGSLISAANNPSLKAAAPQAPWESSTNESSVTVPTLIFACENDSIAPVNSSALPIYD
SMSRNAKQFLEINGGSHSCANSGNSNQALIGKKGVAWMKREMDNDTRYSTFACENPNSTRVSDE
RTANCS
SEQ ID NO: 11: Trx-S93H mutant
MSDKIIHLTDDSFDTDVLKADGAILVDFWAEWCGPCKMIAPILDEIADEYQGKLTVAKLNIDQN
PGTAPKYGIRGIPTLLLFKNGEVAATKVGALSKGQLKEFLDANLAGSGSGHMHHHHHHSSGLVP
RGSGMKETAAAKFERQHMDSPDLGTDDDDKAMAISDPNPYARGPNPTAASLEASAGPFTVRSFT
VSRPSGYGAGTVYYPTNAGGTVGAIAIVPGYTARQSSIKWWGPRLASHGFVVITIDTNSTLDQP
HSRSSQQMAALRQVASLNGTSSSPIYGKVDTARMGVMGWSMGGGGSLISAANNPSLKAAAPQAP
WDSSTNESSVTVPTLIFACENDSIAPVNSSALPIYDSMSRNAKQFLEINGGSHSCANSGNSNQA
LIGKKGVAWMKREMDNDTRYSTFACENPNSTRVSDERTANCS

### REFERENCES

Arza, C. R., Wang, P., Linares□Pastén, J., & Zhang, B. (2019). Synthesis, thermal, rheological characteristics, and enzymatic degradation of aliphatic polyesters with lignin□based aromatic pendant groups. Journal of Polymer Science Part A: Polymer Chemistry, 57(23), 2314-2323.
Berendsen H., Postma J., van Gunsteren W, DiNola A. and Haak J. R., J. Chem. Phys., 1984, 81, 3684-3690
Case V., Babin D., Berryman J., Betz R., Cai Q., Cerutti D., Cheatham T., Darden T., Duke R., Gohlke H., Goetz A., Gusarov S., Homeyer N., Janowski P., Kaus J., Kolossváry I., Kovalenko A., Lee T., LeGrand S., Luchko T., Luo R., Madej B., Merz K., F. Paesani, D. Roe, A. Roitberg, C. Sagui, R. Salomon-Ferrer, G. Seabra, C. Simmerling, Smith W., Swails J., Walker R., Wang J., Wolf R., Wu X. and Kollman P., University of California, 2014 (Computer program).
Darden T., York D. and Pedersen L., J. Chem. Phys., 1993, 98, 10089-10092.
Han, X., Liu, W., Huang, J. W., et al. (2017). Structural insight into catalytic mechanism of PET hydrolase. Nature communications, 8(1), 2106.
Krieger E. and Vriend G., Bioinformatics, 2014, 30, 2981-2982.
Joo, S., Cho, I. J., Seo, H., et al. (2018). Structural insight into molecular mechanism of poly (ethylene terephthalate) degradation. Nature communications, 9(1), 1-12.
Law, K. L. (2017). Plastics in the marine environment. Annual review of marine science, 9, 205-229.
Mark P. and Nilsson L., J. Phys. Chem. A, 2001, 105, 9954-
Shirke, A. N., White, C., Englaender, J. A., et al. (2018). Stabilizing leaf and branch compost cutinase (LCC) with glycosylation: Mechanism and effect on PET hydrolysis. Biochemistry, 57(7), 1190-1200.
Son, H. F., Cho, I. J., Joo, S., et al. (2019). Rational protein engineering of thermo-stable PETase from Ideonella sakaiensis for highly efficient PET degradation. ACS Catalysis, 9(4), 3519-3526.
Wang, P., Linares-Pasten, J. A., & Zhang, B. (2020). Synthesis, Molecular Docking Simulation, and Enzymatic Degradation of AB-Type Indole-Based Polyesters with Improved Thermal Properties. Biomacromolecules, 21(3), 1078-1090.
Yoshida, S., Hiraga, K., Takehana, T., et al. (2016). A bacterium that degrades and assimilates poly (ethylene terephthalate). Science, 351(6278), 1196-1199.

### Items

The present disclosure is described in part by the items below.
1. A polypeptide comprising or consisting of the amino acid sequence of SEQ ID NO: 1,

(SEQ ID NO: 1)
MNPYARGPNPTAASLEASAGPFTVRSFTVSRPSGYGAGTVYYPTNAGGT
VGAIAIVPGYTARQSSIKWWGPRLASHGFVVITIDTNSTLDQPX¹SRSS
QQMAALRQVASLNGTSSSPIYGKVDTARMGVMGWSMGGGGSLISAANNP
SLKAAAPQAPWXDSTNFSSVTVPTLIFACENDSIAPVNSSALPIYDSMS
RNAKQFLEINGGSHSCANSGNSNQALIGKKGVAWMKRFMDNDTRYSTFA
CENPNSTRVSDFRTANCS,

or a sequence having at least 70% identity to SEQ ID NO: 1,
wherein X¹ is a hydrogen bond-donating amino acid, and
wherein the polypeptide is capable of degrading polyester.
2. The polypeptide of item 2, wherein the polypeptide comprises or consists of the amino acid sequence of SEQ ID NO: 1,

(SEQ ID NO: 1)
MNPYARGPNPTAASLEASAGPFTVRSFTVSRPSGYGAGTVYYPTNAGGT
VGAIAIVPGYTARQSSIKWWGPRLASHGFVVITIDTNSTLDQPX¹SRSS
QQMAALRQVASLNGTSSSPIYGKVDTARMGVMGWSMGGGGSLISAANNP
SLKAAAPQAPWX²SSTNFSSVTVPTLIFACENDSIAPVNSSALPIYDSM
SRNAKQFLEINGGSHSCANSGNSNQALIGKKGVAWMKRFMDNDTRYSTF
ACENPNSTRVSDFRTANCS,

or a sequence having at least 70% identity to SEQ ID NO: 1,
wherein X¹ is (R), (K), or (H), and
wherein the side chain of the residue at position X² is negatively charged, and
wherein the polypeptide is capable of degrading polyester.
3. The polypeptide according to any one of the preceding items, wherein the sequence is having at least 75% identity to SEQ ID NO: 1, such as at least 80% identity to SEQ ID NO: 1, such as at least 85% identity to SEQ ID NO: 1, such as at 90% identity to SEQ ID NO: 1, such as at least 95% identity to SEQ ID NO: 1, such as at least 96% identity to SEQ ID NO: 1, such as at least 97% identity to SEQ ID NO: 1, such as at least 98% identity to SEQ ID NO: 1, such as at least 99% identity to SEQ ID NO: 1.
4. The polypeptide according to any one of the preceding items, wherein the polypeptide comprises or consists of an amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10.
5. The polypeptide according to any one of the preceding items, wherein X¹ is (R).
6. The polypeptide according to any one of the preceding items, wherein X¹ is (K).
7. The polypeptide according to any one of the preceding items, wherein X¹ is (H).
8. The polypeptide according to any one of the preceding items, wherein X² is (E) or D.
9. The polypeptide according to any one of the preceding items, wherein X² is (D).
10. The polypeptide according to any one of the preceding items, wherein the distance between at least one atom in X¹ and at least one atom in X² is less than or equal to 5.0 Å.
11. The polypeptide according to any one of the preceding items, wherein X¹ and X² are capable of interacting with each other.
12. The polypeptide according to item 11, wherein the interaction is a charge-charge interaction.
13. The polypeptide according to item 12, wherein the charge-charge interaction is a salt bridge.
14. The polypeptide according to any one of the preceding items, wherein the polypeptide is conjugated to thioredoxin.
15. The polypeptide according to item 14, wherein thioredoxin is conjugated to the N-terminal end of the polypeptide.
16. The polypeptide according to item 14, wherein thioredoxin is conjugated to the C-terminal end of the polypeptide.
17. A composition comprising the polypeptide according to any one of the preceding items.
18. The composition according to item 17, wherein the composition is an aqueous buffer.
19. A polynucleotide encoding the polypeptide according to any one of the preceding items.
20. A vector comprising the polynucleotide according to item 19.
21. A cell comprising the polynucleotide according to item 19, or the vector according to item 20.
22. A method of degrading polyethylene terephthalate, the method comprising contacting the polypeptide according to any one of the preceding items with a compound or composition comprising polyethylene terephthalate, thus degrading the polyethylene terephthalate.
23. The method according to item 22, wherein the degradation of the polyethylene terephthalate is hydrolysis of the polyethylene terephthalate.
24. The method according to item 22, wherein the composition or compound is a composite material.
25. The method according to item 24, wherein the composite material comprises at least one other polymer than polyethylene terephthalate.
26. A method of manufacturing terephthalic acid comprising the steps of:
   a. providing the polypeptide according to any one of items Error! Reference source not found. to 16,
   b. providing a material comprising polyethylene terephthalate,
   c. contacting the polypeptide of step (a) and the material comprising polyethylene terephthalate of (b), thus forming a mixture comprising terephthalic acid, and
   d. extracting terephthalic acid from said mixture,
   thus manufacturing terephthalic acid.
27. A method of manufacturing ethylene glycol comprising the steps of:
   a. providing the polypeptide according to any one of items Error! Reference source not found. to 16,
   b. providing a material comprising polyethylene terephthalate,
   c. contacting the polypeptide of step (a) and the material comprising polyethylene terephthalate of (b), thus forming a mixture comprising ethylene glycol, and
   d. extracting ethylene glycol from said mixture,
   thus manufacturing ethylene glycol.
28. The method according to any one of items 26 and 27, further comprising a step wherein the mixture from step (c) is heated.
29. The method according to any one of items 17 to 28, wherein the polypeptide is capable of degrading polyester.
30. The method according to any one of items Error! Reference source not found. to 29, wherein the polypeptide is capable of degrading polyethylene terephthalate.
31. The method according to any one of items Error! Reference source not found. to 30, wherein the polypeptide is capable of degrading a polymeric substrate at a temperature of at least 45° C., such as at least 50° C., such as at least 55° C., such as at least 58° C., such as least 60° C.
32. The method according to any one of items Error! Reference source not found. to 31, wherein the polypeptide is capable of degrading polyethylene terephthalate at a temperature of 60° C.
33. The method according to any one of items Error! Reference source not found. to 32, wherein the Tₘ of said polypeptide is at least 55° C.
34. The method according to any one of items Error! Reference source not found. to 33, wherein the polypeptide is stable in a solution for at least 5 months at 4° C.
35. A polypeptide comprising or consisting of the amino acid sequence of SEQ ID NO: 1,

(SEQ ID NO: 1)
MNPYARGPNPTAASLEASAGPFTVRSFTVSRPSGYGAGTVYYPTNAGGT
VGAIAIVPGYTARQSSIKWWGPRLASHGFVVITIDTNSTLDQPX¹SRSS
QQMAALRQVASLNGTSSSPIYGKVDTARMGVMGWSMGGGGSLISAANNP
SLKAAAPQAPWX²SSTNFSSVTVPTLIFACENDSIAPVNSSALPIYDSM
SRNAKQFLEINGGSHSCANSGNSNQALIGKKGVAWMKRFMDNDTRYSTF
ACENPNSTRVSDFRTANCS,

or a sequence having at least 70% identity to SEQ ID NO: 1,
wherein X¹ is cysteine (C), and
wherein X² is cysteine (C) and wherein the polypeptide is capable of degrading polyester.
36. The polypeptide according to item 35, wherein the cysteine at X¹ and X² are capable of forming a disulphide bridge.

1. A polypeptide comprising or consisting of the amino acid sequence of SEQ ID NO: 1,
(SEQ ID NO: 1)
MNPYARGPNPTAASLEASAGPFTVRSFTVSRPSGYGAGTVYYPTNAGGT
VGAIAIVPGYTARQSSIKWWGPRLASHGFVVITIDTNSTLDQPX¹SRSS
QQMAALRQVASLNGTSSSPIYGKVDTARMGVMGWSMGGGGSLISAANNP
SLKAAAPQAPWX²SSTNFSSVTVPTLIFACENDSIAPVNSSALPIYDSM
SRNAKQFLEINGGSHSCANSGNSNQALIGKKGVAWMKRFMDNDTRYSTF
ACENPNSTRVSDFRTANCS,
or a sequence having at least 70% identity to SEQ ID NO: 1,
wherein X¹ is arginine (R), lysine (K), or histidine (H), and
wherein the side chain of the residue at position X² is negatively charged, and wherein the polypeptide is capable of degrading polyester.

2. The polypeptide according to claim 1, wherein the sequence is having at least 75% identity to SEQ ID NO: 1, or at least 80% identity to SEQ ID NO: 1, or at least 85% identity to SEQ ID NO: 1, or at 90% identity to SEQ ID NO: 1, or at least 95% identity to SEQ ID NO: 1, or at least 96% identity to SEQ ID NO: 1, or at least 97% identity to SEQ ID NO: 1, or at least 98% identity to SEQ ID NO: 1, or at least 99% identity to SEQ ID NO: 1.

3. The polypeptide according to claim 1, wherein the polypeptide comprises or consists of an amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11.

4. The polypeptide according to claim 1, wherein X¹ is arginine (R).

5. The polypeptide according to claim 1, wherein X¹ is lysine (K).

6. The polypeptide according to claim 1, wherein X¹ is histidine (H).

7. The polypeptide according to claim 1, wherein X² is glutamic acid (E) or aspartic acid (D).

8. The polypeptide according to claim 1, wherein the distance between at least one atom in X¹ and at least one atom in X² is less than or equal to 5.0 Å.

9. The polypeptide according to claim 1, wherein X¹ and X² are capable of interacting with each other.

10. The polypeptide according to claim 9, wherein the interaction is a charge-charge interaction and/or a salt bridge.

11. (canceled)

12. The polypeptide according to claim 1, wherein the polypeptide is conjugated to thioredoxin.

13. The polypeptide according to claim 12, wherein thioredoxin is conjugated to the N-terminal end of the polypeptide or to the C-terminal end of the polypeptide.

14. (canceled)

15. The polypeptide according to claim 1, wherein said polypeptide has higher thermostability, and/or higher enzymatic activity compared to the wild-type polypeptide.

16. (canceled)

17. The polypeptide according to claim 1, wherein said polypeptide has higher enzymatic activity at temperatures higher than room temperature compared to the wild-type polypeptide, optionally wherein said polypeptide has a stable enzymatic activity at temperatures in the range of 25° C. to 85° C.

18. (canceled)

19. (canceled)

20. (canceled)

21. A polynucleotide encoding the polypeptide according to claim 1.

22. (canceled)

23. (canceled)

24. A method of degrading polyethylene terephthalate, the method comprising contacting the polypeptide according to claim 1 with a compound or composition comprising polyethylene terephthalate, thus degrading the polyethylene terephthalate.

25. (canceled)

26. (canceled)

27. (canceled)

28. A method of manufacturing terephthalic acid and/or ethylene glycol comprising the steps of:
(a) providing the polypeptide according to claim 1,
(b) providing a material comprising polyethylene terephthalate,
(c) contacting the polypeptide of step (a) and the material comprising polyethylene terephthalate of (b), thus forming a mixture comprising terephthalic acid and/or ethylene glycol, optionally wherein the mixture is heated, and
(d) extracting terephthalic acid and/or ethylene glycol from said mixture,
thus manufacturing terephthalic acid and/or ethylene glycol.

29. (canceled)

30. (canceled)

31. The method according to claim 28, wherein the polypeptide is capable of degrading polyester and/or polyethylene terephthalate.

32. (canceled)

33. (canceled)

34. (canceled)

35. (canceled)

36. (canceled)

37. A polypeptide comprising or consisting of the amino acid sequence of SEQ ID NO: 1,
(SEQ ID NO: 1)
MNPYARGPNPTAASLEASAGPFTVRSFTVSRPSGYGAGTVYYPTNAGGT
VGAIAIVPGYTARQSSIKWWGPRLASHGFVVITIDTNSTLDQPX¹SRSS
QQMAALRQVASLNGTSSSPIYGKVDTARMGVMGWSMGGGGSLISAANNP
SLKAAAPQAPWX²SSTNFSSVTVPTLIFACENDSIAPVNSSALPIYDSM
SRNAKQFLEINGGSHSCANSGNSNQALIGKKGVAWMKRFMDNDTRYSTF
ACENPNSTRVSDFRTANCS,
or a sequence having at least 70% identity to SEQ ID NO: 1,
wherein X¹ is cysteine (C), and
wherein X² is cysteine (C) and wherein the polypeptide is capable of degrading polyester.

38. The polypeptide according to claim 37, wherein the cysteine at X¹ and X² are capable of forming a disulphide bridge.
